(19) United States
(12) Patent Application Publication
(10) Pub. No.: US 2021/0087155 A1
(43) Pub. Date: Mar. 25, 2021

(54) AN IMPROVED PROCESS FOR THE PREPARATION OF VORTIOXETINE AND SALTS THEREOF

(71) Applicant: PIRAMAL ENTERPRISES LIMITED, Mumbai (IN)

(72) Inventors: Milind GHARPURE, Mumbai, Maharashtra (IN); Sanjay Kumar SHARMA, Mumbai, Maharashtra (IN); Nainesh KANSAGARA, Mumbai, Maharashtra (IN); Yogesh ZALTE, Mumbai, Maharashtra (IN); Pravin THOMBRE, Mumbai, Maharashtra (IN); Sudha MENON, Mumbai, Maharashtra (IN)

(73) Assignee: PIRAMAL ENTERPRISES LIMITED, Mumbai (IN)

(21) Appl. No.: 16/967,640

(22) PCT Filed: Feb. 5, 2019
(86) PCT No.: PCT/IB2019/050889

§ 371 Date: Aug. 5, 2020

(30) Foreign Application Priority Data
Feb. 6, 2018 (IN) 201821004449

(51) Int. Cl. C07D 295/033

(52) U.S. Cl. CPC C07D 295/033; C07B 2200/13

(57) ABSTRACT

The present invention relates to a novel crystalline polymorphic form of 1-[2-(2,4-dimethyl-phenylsulfanyl)-phenyl]-piperazine hydrochloride; commonly known as vortioxetine hydrochloride (hereafter referred to as the compound (Ia) and process for its preparation comprising of treating the compound (Ia) (as described herein) with a ketone solvent or mixture of ketone solvent with other solvents. The present invention also relates to an improved process for the preparation of vortioxetine hydrobromide (Ia), comprising reacting the compound (I) (as described herein) with hydrogen bromide solution in acetic acid.

### FIELD OF THE INVENTION

The present invention relates to a novel crystalline polymorphic form of 1-[2-(2,4-dimethyl-phenylsulfanyl)-phenyl]-piperazine hydrochloride; commonly known as vorioxetine hydrochloride (Ia) and process for its preparation. The present invention also relates to an improved process for the preparation of vortioxetine hydrobromide (Ia).

### BACKGROUND OF THE INVENTION

The following discussion of the prior art is intended to present the invention in an appropriate technical context, and allows its significance to be properly appreciated. Unless clearly indicated to the contrary, reference to any prior art in this specification should not be construed as an expressed or implied admission that such art is widely known or forms part of common general knowledge in the field.

Vortioxetine of formula (I) is a typical antidepressant indicated for the treatment of major depressive disorder and marketed under the brand name as TRINTELLX. The marketed compound is in the form of its hydrobromide salt which is chemically known as 1-[2-(2,4-dimethyl-phenylsulfanyl)-phenyl]-piperazine, hydrobromide (Ia).

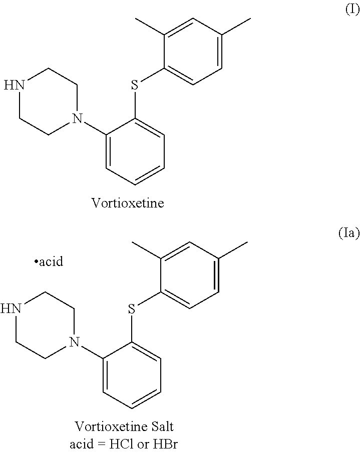 [SMILES: Cc1ccc(Sc2ccccc2N2CCNCC2)c(C)c1 ; Cc1ccc(Sc2ccccc2N2CCNCC2)c(C)c1]

Vortioxetine being an important antidepressant agent; a number of processes for its preparation as well as for its intermediates are known in the art.

U.S. Pat. No. 8,722,684 refers to the method for preparation of hydrobromide salt of vortioxetine. The process consists of dissolving 1-[2-(2,4-Dimethylphenylsulfanyl)-phenyl]piperazine in hot ethyl acetate followed by the treatment with 48% aqueous HBr solution to obtain vortioxetine hydrobromide.

U.S. Pat. No. 9,493,409 describes a process for the preparation of vortioxetine consisting of reacting 2-((2,4-dimethylphenyl)thio)aniline with bis(2-chloroethyl)amine hydrochloride in the presence of diethylene glycol methyl ether at 130° C. for 3 days to provide the product as vortioxetine hydrochloride. The obtained vortioxetine hydrochloride was further treated with sodium hydroxide followed by 48% hydrobromic acid solution (48% HBr) to obtain the corresponding compound as vortioxetine hydrobromide.

Published PCT application WO 2016/004908 A1 describes a process for the preparation of 1-(2-(2,4-dimethylphenylsulphanyl)phenyl)piperazine hydrochloride comprising reaction of 2-(2,4-dimethylphenyl sulphanyl) benzeneamine with a suitable precursor of formation of piperazine ring in an aromatic solvent selected from the group consisting of chlorobenzene, xylene, toluene, α,α,α-trifluorotoluene and their mixtures. In addition to the afore-mentioned patent documents, there are a number of patent documents that describe a process for the preparation of vortioxetine, its intermediates and salts thereof.

For instance, published PCT application WO2015114395 and WO2014161976A1; published US patent application 2016/0060215; Chinese patent applications CN 103788020, CN 103936694, CN 104109135, CN104098530; U.S. Pat. No. 9,095,588 B2 describes a process for the preparation of vortioxetine and its salts.

It is evident from the discussion of the processes for the preparation of vortioxetine and its salts, described in the afore cited patent documents that the reported processes provide a product with low yield and purity; and requires repeated purification or multiple crystallization steps. Also, the prior art process requires reactions of prolonged duration such as overnight stirring; treatment of the compound with hot solvents during salt formation; which renders the process hazardous and costlier, hence are not industrially feasible. In view of these drawbacks, there is a need to develop an industrially viable commercial process for the preparation of vortioxetine and its salts; which is a simple, efficient and cost-effective process and provides the desired compounds in improved yield and purity.

Further. U.S. Pat. No. 8,722,684 refers to the phenylsulfanyl-piperazine compounds, wherein the specification disclosed crystalline polymorphic forms for various salts of Vortioxetine. Accordingly, the patent US'684 disclosed the crystalline form of Vortioxetine hydrochloride to with selected X-ray peak positions (° 2θ) as 9.41, 12.37, 19.66 and 22.55. Similarly, the patent US'684 disclosed the crystalline form of Vortioxetine hydrochloride monohydrate with selected X-ray peak positions (° 2θ) as 7.72, 13.45, 15.39 and 17.10.

Chinese patent applications CN 105153066 and CN 105111167 disclosed new crystalline forms of vortioxetine hydrochloride and process for its preparation.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predictable solubility profiles. It is desirable to investigate all solid-state forms of an active drug substance, including all polymorphic forms, and to determine the stability, dissolution and flow properties of each polymorphic form. The polymorphic forms of a compound can be distinguished in a laboratory by X-ray diffraction spectroscopy and by other methods such as, infrared spectrometry.

Hence, there is continuous scope to develop alternate novel crystalline form of Vortioxetine hydrochloride with enhanced stability and product quality.

Inventors of the present invention have developed an improved process that addresses the problems associated with the processes reported in the prior art. The process of the present invention does not involve use of any toxic and/or costly solvents, also does not involve use of costlier coupling agents and reagents. Moreover, the process does not require repetitive purification steps. Accordingly, the present invention provides a process for the preparation of vortioxetine, which is simple, efficient, cost effective, environmentally friendly and commercially scalable for large scale operations.

### SUMMARY OF THE INVENTION

In one aspect, the present invention relates to an improved process for the preparation of vortioxetine hydrobromide (Ia), comprising reacting the compound (I) (as described herein) with hydrogen bromide solution in acetic acid.

In an embodiment, there is provided a novel crystalline ‘Form-P’ of vortioxetine hydrochloride (Ia).

In a yet another embodiment, there is provided a novel crystalline ‘Form-P’ of vortioxetine hydrochloride (a) having characteristic X-ray powder diffraction (° 2θ) angle 3.82, 7.66, 11.48, 19.20, 20.23 and 23.1.

In a yet another embodiment, there is provided a novel crystalline Form-P of vortioxetine hydrochloride (Ia) having characteristic X-ray powder diffraction (d spacing) 23.10, 11.53, 7.70, 4.62, 4.39 and 3.85.

In another aspect, the present invention relates to a process for the preparation of vorioxetine hydrochloride (the compound (I).HCl) crystalline Form-P, comprising treating the compound (Ia) with a ketone solvent or mixture of ketone solvent in other solvents.

### DESCRIPTION OF THE FIGURES

FIG. 1: X-ray Powder Diffraction (XRPD) pattern of the vortioxetine hydrochloride ‘Form-P’.

### DETAILED DESCRIPTION OF THE INVENTION

Accordingly, the present invention relates to an improved process for the preparation of vortioxetine hydrobromide (Ia), represented by the following formula,

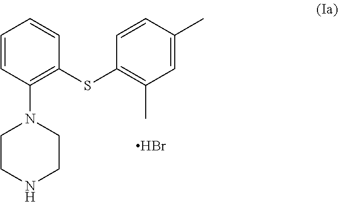 [SMILES: Cc1ccc(Sc2ccccc2N2CCNCC2)c(C)c1 ; Br]

comprising reacting the vortioxetine (the compound (I) represented by the following formula;

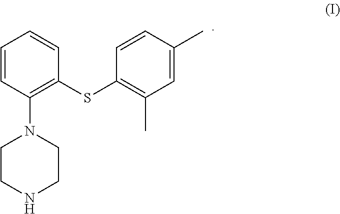 [SMILES: Cc1ccc(Sc2ccccc2N2CCNCC2)c(C)c1]

with hydrogen bromide solution in acetic acid.

Accordingly, there is provided an improved process for the preparation of vortioxetine hydrobromide (Ia), represented by the following formula,

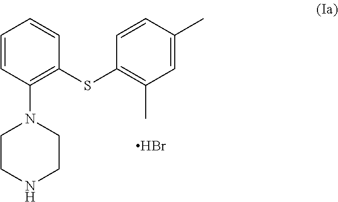 [SMILES: Cc1ccc(Sc2ccccc2N2CCNCC2)c(C)c1 ; Br]

comprising the step of.
(a) optionally, dissolving the vortioxetine (D represented by the following formula; in an organic solvent;

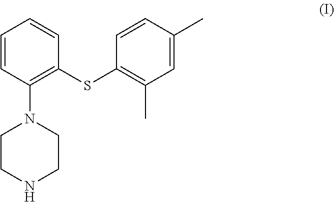 [SMILES: Cc1ccc(Sc2ccccc2N2CCNCC2)c(C)c1]

(b) treating the reaction mixture containing vortioxetine (D of stage (a) with hydrogen bromide solution in acetic acid.

In the context of the present invention, the term “optionally” when used in reference to any element; including a process step e.g. dissolving the compound in organic solvent; it is intended to mean that the subject element that is ‘organic solvent’ is used, or alternatively, is not used in the reaction. Both alternatives are intended to be within the scope of the present invention.

In an embodiment, the organic solvent is selected from the group consisting of the halogenated solvent such as dichloromethane, 4-bromotoluene, di-iodomethane, carbon tetrachloride, chlorobenzene and chloroform; alcoholic solvent such as methanol, ethanol, isopropanol, t-amyl alcohol, t-butyl alcohol and hexanol; an ether solvent such as tetrahydrofuran, cyclopentyl methyl ether, 2-methyltetrahydrofuran, diethyl ether and 1,4-dioxane; a ketone selected from methyl ethyl ketone, acetone, methyl isobutyl ketone (MIBK); an aprotic solvent such as acetonitrile, N,N-dimethyl formamide (DMF), N,N-dimethyl acetamide, dimethyl sulfoxide (DMSO) and N-methylpyrrolidone (NMP); an aromatic solvent such as toluene, xylene and benzene; acetone; or a mixture thereof.

In a specific embodiment, the process for the preparation of vortioxetine hydrobromide (Ia), comprises the steps of:
(1) dissolving the vortioxetine (I) in an organic solvent;
(2) adding the hydrogen bromide solution in acetic acid to the reaction mixture of step (1);
(3) heating the reaction mixture of the above step (2) at higher temperature of about 40-100° C.;
(4) cooling the reaction mixture of the above step (3) at temperature of about 30° C.;
(5) isolating the product vortioxetine hydrobromide (Ia).

The process of the present invention as per the specific embodiment described above is illustrated in the following Scheme-1.

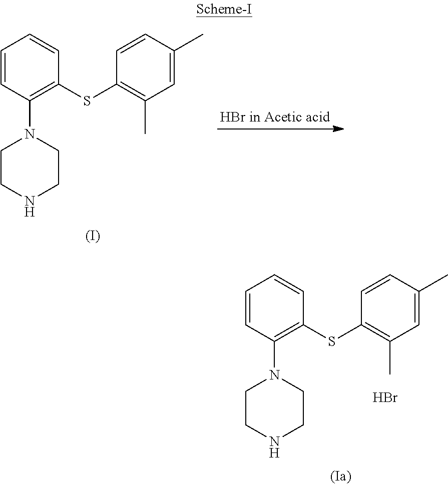 [SMILES: Cc1ccc(Sc2ccccc2N2CCNCC2)c(C)c1 ; "(I)" ; Cc1ccc(Sc2ccccc2N2CCNCC2)c(C)c1 ; Br]

In an embodiment, the reaction is performed at a temperature ranging between 40−100° C., preferably 50-80° C.,

The organic solvent used in the step-(1) of the above process (as depicted in the Scheme-I) is selected from the group consisting of the halogenated solvent such as dichloromethane, 4-bromotoluene, diiodomethane, carbon tetrachloride, chlorobenzene and chloroform; alcoholic solvent such as methanol, ethanol, isopropanol, t-amyl alcohol, t-butyl alcohol and hexanol; an ether solvent such as tetrahydrofuran, cyclopentyl methyl ether, 2-methyltetrahydrofuran, diethyl ether and 1,4-dioxane; a ketone selected from methyl ethyl ketone, acetone, methyl isobutyl ketone (MIBK); an aprotic solvent such as acetonitrile, N,N-dimethyl formamide (DMF), N,N-dimethyl acetamide, dimethyl sulfoxide (DMSO) and N-methylpyrrolidone (NMP); an aromatic solvent such as toluene, xylene and benzene; acetone; or a mixture thereof.

The term ‘higher temperature of about 40-100° C.’ referred to in the step (3) of the above process (as depicted in the Scheme-I) can range from 45° C. to 90° C., preferably 50-80° C.

The term ‘temperature of about 30° C.’ referred to in the step (4) of the above process (as depicted in the Scheme-I) can range from 25° C. to 35° C.

The term ‘isolating the product’ referred to in the step (5) corresponds to the steps involving separation of organic phase, filtration, evaporation of solvent, washing and drying; precipitation, filtration of precipitated product.

The process of the present invention as illustrated in the above Scheme-I comprise treating vortioxetine hydrochloride (a) with sodium hydroxide solution (NaOH) to obtain vortioxetine (I) freebase crude. Vortioxetine (I) free base was dissolved in acetone followed by the addition of HBr in acetic acid solution into the reaction mass. The reaction mixture was stirred for 1 h at about 50° C. temperature; to provide the desired product % yield-about 85-90% and more than 99% HPLC purity.

It is evident from the processes reported in the prior art that the vortioxetine hydrobromide was obtained with low yield of about 80%; whereas the process of the present invention provided the pure vortioxetine in a yield of about 85-90% and purity of more than about 99% (HPLC). It is further evident that the prior art process refers the use of harsh reaction conditions, prolonged reaction time and requires repeated purification or multiple crystallization steps; which are avoided in the currently presented improved process. This amounts to a significant advantage over the processes reported in the prior art.

Advantageously, the above identified elements of the process of the instant invention effectively contribute to the reduction of overall cost of the process.

In an embodiment, there is provided a novel crystalline ‘Form-P’ of vortioxetine hydrochloride (Ia).

In another embodiment, there is provided a novel crystalline Form-P of vortioxetine hydrochloride (Ia) having characteristic X-ray powder diffraction (° 2θ) angle 3.82, 7.66, 11.48, 19.20, 20.23 and 23.1.

In a yet another embodiment, there is provided a novel crystalline Form-P of vortioxetine hydrochloride (Ia) having characteristic X-ray powder diffraction (d spacing) 23.10, 11.53, 7.70, 4.62, 4.39 and 3.85.

In an embodiment, the crystalline Form-P of the vortioxetine hydrochloride (Ia) is further characterized by the X-ray powder diffraction graph having d-spacing and 2-theta values as per Table−1.

The X-ray powder diffraction spectrum of vortioxetine hydrochloride (Ia) was measured under the following experimental conditions;

Instrument X-Ray Diffractometer. Bruker D8 Advance

X-Ray: Cu/40 kv/40 mA

Diverging 0.3°

Counter Lynx Eye

Scan Mode Continuous

Scan Axes: Two Theta/Theta

Scan Range: 3° to 40°

Generator power: 40 kV 40 mA

Scan range 3-40° 2 θ

Step size 0.02°

Step time: 0.25 se

Sample rotation: 15 rpm

Detector Lynx-Eye

TABLE 1
summarizes the d-spacing values in °A, and the corresponding 2θ values
of the crystalline Form-P of the vortioxetine hydrochloride (Ia).

| Angle | d value | |
|---|---|---|
| 2-Theta ° | Angstrom | Intensity % |
| 3.82 | 23.10 | 100.00 |
| 7.66 | 11.53 | 45.80 |
| 11.48 | 7.70 | 34.00 |
| 13.36 | 6.62 | 1.70 |
| 13.78 | 6.42 | 7.20 |
| 14.41 | 6.14 | 1.00 |
| 14.91 | 5.94 | 1.10 |
| 15.32 | 5.78 | 9.20 |
| 15.93 | 5.56 | 1.00 |
| 16.35 | 5.42 | 2.20 |
| 17.02 | 5.21 | 3.10 |
| 17.60 | 5.03 | 8.40 |
| 18.30 | 4.84 | 5.80 |
| 18.84 | 4.71 | 3.60 |
| 19.20 | 4.62 | 49.90 |
| 20.23 | 4.39 | 19.50 |
| 21.40 | 4.15 | 3.40 |
| 21.81 | 4.07 | 4.10 |
| 22.85 | 3.89 | 3.80 |
| 23.10 | 3.85 | 59.50 |
| 23.38 | 3.80 | 4.10 |
| 24.02 | 3.70 | 1.90 |
| 24.35 | 3.65 | 4.90 |
| 25.02 | 3.56 | 4.50 |
| 25.50 | 3.49 | 2.40 |
| 26.69 | 3.34 | 2.70 |
| 26.99 | 3.30 | 3.80 |
| 27.55 | 3.23 | 11.90 |
| 27.99 | 3.19 | 1.30 |
| 28.55 | 3.12 | 9.80 |
| 29.24 | 3.05 | 2.60 |
| 30.00 | 2.98 | 1.50 |
| 30.17 | 2.96 | 2.60 |
| 30.93 | 2.89 | 3.10 |
| 31.87 | 2.81 | 4.40 |
| 32.70 | 2.74 | 3.00 |
| 33.59 | 2.67 | 3.00 |
| 34.08 | 2.63 | 1.10 |
| 34.97 | 2.56 | 10.50 |
| 35.22 | 2.55 | 1.70 |
| 35.57 | 2.52 | 2.10 |
| 36.27 | 2.48 | 1.20 |
| 37.16 | 2.42 | 1.50 |
| 37.51 | 2.40 | 1.80 |
| 38.16 | 2.36 | 3.00 |
| 38.35 | 2.35 | 2.20 |
| 38.98 | 2.31 | 4.80 |
| 39.83 | 2.26 | 2.00 |

In another embodiment, the present invention relates to a process for the preparation of vortioxetine hydrochloride (Ia) crystalline Form-P, comprising treating the compound (Ia) (as described herein) with a ketone solvent or mixture of ketone solvent in other solvents.

Accordingly, the present invention relates to an improved process for the preparation of vortioxetine hydrochloride (a) crystalline Form-P, represented by the following formula,

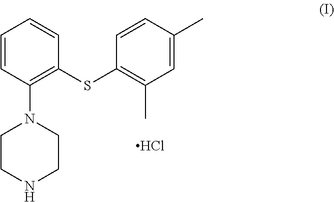 [SMILES: Cc1ccc(Sc2ccccc2N2CCNCC2)c(C)c1 ; Cl]

comprising treating vortioxetine hydrochloride (Ia) with a ketone solvent or mixture of ketone solvent with other solvents.

In a specific embodiment, the process for the preparation of vortioxetine hydrochloride (Ia) crystalline Form-P, comprises the steps of:
(i) suspending the vortioxetine hydrochloride (Ia) in a ketone solvent or mixture of ketone solvent with other solvents;
(ii) stirring the reaction mixture of step (i);
(iii) isolating the product vortioxetine hydrochloride crystalline Form-P.

The ketone solvent used in the step-(i) of the above process is selected from the group consisting of methyl ethyl ketone, acetone, methyl isobutyl ketone (MIBK) and the like.

The other solvent used in the step-(i) of the above process is selected from the group consisting of the halogenated solvent such as dichloromethane, 4-bromotoluene, diiodomethane, carbon tetrachloride, chlorobenzene and chloroform; alcoholic solvent such as methanol, ethanol, isopropanol, t-amyl alcohol, t-butyl alcohol and hexanol; an ether solvent such as tetrahydrofuran, cyclopentyl methyl ether, 2-methyltetrahydrofuran, diethyl ether and 1,4-dioxane; a ketone selected from methyl ethyl ketone, acetone, methyl isobutyl ketone (MIBK); an aprotic solvent such as acetonitrile, N,N-dimethyl formamide (DMF), N,N-dimethyl acetamide, dimethyl sulfoxide (DMSO) and N-methylpyrolidone (NMP); an aromatic solvent such as toluene, xylene and benzene; acetone; water or a mixture thereof.

The term ‘isolating the product’ referred to in the step (iii) corresponds to the one or more is steps involving separation of organic phase, filtration, evaporation of solvent, precipitation, filtration of precipitated product, washing and drying.

There are several prior art documents that disclosed the methods for preparation of vortioxetine (I) fee base such as U.S. Pat. Nos. 7,144,884; 9,493,409; Published PCT application WO 2016/004908 A1 and other. The process in general comprises reaction of 2-((24-dimethylphenyl)thio)aniline with bis(2-chloroethyl)amine or its salt in varying solvent and reaction conditions to obtain vortioxetine free base. Any of the prior art method can be adopted for the purpose of preparation of vortioxetine (I) free base.

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### EXAMPLES

### Example-1: Preparation of Vortioxetine Hydrochloride (‘Form-P’) [Ia]

Charged 750 mL of N-methyl-2-pyrrolidone (NMP) in a flask followed by the addition of 2-((2,4-dimethylphenyl)thio)aniline (250 g), Bis(2-chloroethyl)amine hydrochloride (779.5 g) and catalytic amount of potassium iodide. The reaction mixture was heated at a temperature of 110-120° C. for 2 days. The reaction mixture was cooled to the temperature of 25-30° C. followed by the addition of water (3 L) and Toluene (750 mL). The reaction mixture was cooled to 0-5° C. temperature and the obtained solid was filtered. The wet cake was suspended in acetone (1105 ml) and water (220 mL). The solid obtained was filtered and dried under vacuum as Vortioxetine Hydrochloride (Form-P) [yield: 1.75 g (47.8%): Purity: 99.8% (HPLC)].

### Example-2: Preparation of Vortioxetine hydrochloride (‘Form-P’) [Ia]

Charged 360 mL of N-Methyl-2-pyrrolidone (NMP) in a flask followed by the addition of 2-((2,4-dimethylphenyl)thio)aniline (120 g), Bis(2-chloroethyl)amine hydrochloride (374 g) and catalytic amount of potassium iodide. The reaction mixture was heated at a temperature of is 110-120° C. for 2 days. The reaction mixture was cooled to the temperature of 25-30° C., followed by the addition of water (1440 mL) and Toluene (360 mL). The reaction mixture was cooled to 0-5° C. temperature and the obtained solid was filtered. The wet cake was suspended in acetone (1200 mL). The solid obtained was filtered and dried under vacuum as Vortioxetine Hydrochloride (Form-P) [Yield: 103 g (58.77%); Purity: 99.7% (HPLC)].

### Example-3: Preparation of Vortioxetine Hydrobromide [Ia]

Charged 10 mL of dichloromethane in a flask followed by the addition of Vortioxetine Hydrochloride (2 g), 10 mL of water and sodium hydroxide (0.36 g). The reaction mixture was stirred at a temperature of 25-30° C. and the separate organic layer was distilled out under vacuum. To the residue was added 20 mL of acetone and hydrobromic acid (1.6 g) solution in acetic acid; and the reaction mixture was heated at 50° C. temperature for 1 h. The reaction mixture was cooled to 25-30° C. temperature and the solid obtained was filtered to provide Vortioxetine Hydrobromide [Yield: 1.5 g (66%); Purity: 99.6% (HPLC)].

### Example-4: Preparation of Vortioxetine Hydrobromide [Ia]

Charged 10 ml of dichloromethane in a flask followed by the addition of Vortioxetine Hydrochloride (2 g), 10 mL of water and sodium hydroxide (0.36 g). The reaction mixture was stirred at a temperature of 25-30° C. and the separate organic layer was distilled out under vacuum. To the residue was added 20 mL of acetonitrile and hydrobromic acid (1.6 g) solution in acetic acid; and the reaction mixture was heated at 50° C. temperature for 1 h. The reaction mixture was cooled to 25-30° C. temperature and the solid obtained was filtered to provide Vortioxetine Hydrobromide [Yield: 1.7 g (75%); Purity: 99.9% (HPLC)].

### Example-5: Preparation of Vortioxetine Hydrobromide [Ia]

Charged 10 mL of dichloromethane in a flask followed by the addition of Vortioxeine Hydrochloride (2 g), 10 mL of water and sodium hydroxide (0.36 g). The reaction mixture was stirred at a temperature of 25-30° C. and the separate organic layer was distilled out under vacuum. To the residue was added 20 mL of methyl isobutyl ketone (M3K) and hydrobromic acid (1.6 g) solution in acetic acid; and the reaction mixture was heated at 50° C. temperature for 1 h. The reaction mixture was cooled to 25-30° C. temperature and the solid obtained was filtered to provide Vortioxetine Hydrobromide [Yield: 1.7 g (75%); Purity: 99.88% (HPLC)].

### Example-6: Preparation of Vortioxetine Hydrobromide [Ia]

Charged 10 mL of dichloromethane in a flask followed by the addition of Vortioxetine Hydrochloride (2 g), 10 mL of water and sodium hydroxide (0.36 g). The reaction mixture was stirred at a temperature of 25-30° C. and the separate organic layer was distilled out under vacuum. To the residue was added 20 mL of toluene and hydrobromic acid (1.6 g) solution in acetic acid; and the reaction mixture was heated at 80° C. temperature for 1 h. The reaction mixture was cooled to 25-30° C. temperature and the solid obtained was filtered to provide Vortioxetine Hydrobromide [Yield: 1.5 g (66%); Purity: 99.9% (HPLC)].

We claim:

1) An improved process for the preparation of vortioxetine hydrobromide (Ia), comprising:
a) optionally dissolving the vortioxetine (I) in an organic solvent; and/or
b) treating the resulting solution with hydrogen bromide solution in acetic acid.

2) The process as claimed in claim 1, wherein solvent used in step (a) is selected from ketones such as methyl ethyl ketone, acetone, methyl isobutyl ketone (MIBK): aprotic solvents such as acetonitrile and proprionitrile; aromatic solvent such as toluene, xylene and benzene.

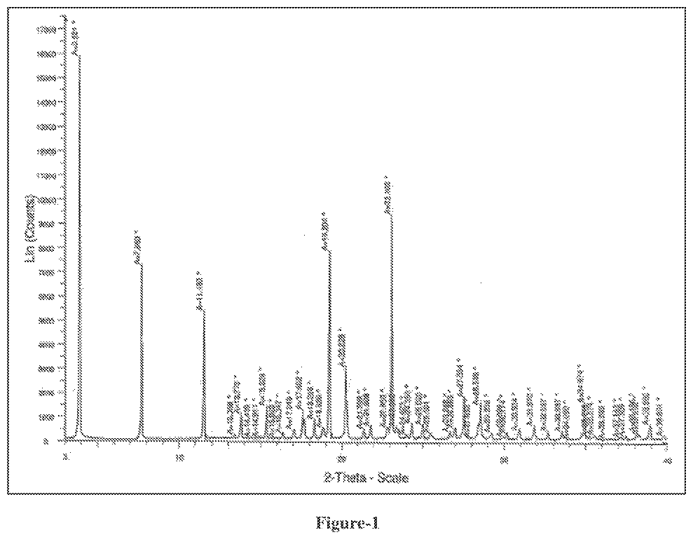

3) Crystalline vortioxetine hydrochloride Form-P characterized by an x-ray diffraction pattern (XRD) as shown in FIG. 1

4) Crystalline vorioxetine hydrochloride Form-P as claimed in claim 3, having characteristic X-ray powder diffraction with peaks at about 3.82, 7.66, 11.48, 19.20, 20.23 and 23.1±0 2 degrees two-theta.

5) Crystalline vortioxetine hydrochloride Form-P as claimed in claim 3, having characteristic X-ray powder diffraction pattern with reflections corresponding to the d-spacing values 23.10, 11.53, 7.70, 4.62, 4.39 and 3.85.

6) A process for the preparation of vortioxetine hydrochloride (Ia) crystalline Form-P, comprises the steps of:
a) suspending the vortioxetine hydrochloride (Ia) in a ketone solvent or mixture of ketone solvent with other solvents; and
b) optionally stirring the reaction mixture of step (a);
c) isolating the product vortioxetine hydrochloride crystalline Form-P.

7) The process as claimed in claim 4, wherein solvent used in step (a) is selected from ketones such as methyl ethyl ketone, acetone, methyl isobutyl ketone (MIBK).

8) The process as claimed in claim 4, wherein other solvent used in step (a) is selected from water.
